(19) United States
(12) Patent Application Publication
(10) Pub. No.: US 2003/0105465 A1
(43) Pub. Date: Jun. 5, 2003

(54) Implant screw and washer assembly and method of fixation

(76) Inventors: Reinhold Schmieding, Naples, FL (US); Peter J. Dreyfuss, Naples, FL (US); Karen L. Gallen, Naples, FL (US); Jeffrey Wyman, Naples, FL (US); Hans Linden, Koln (DE)

Correspondence Address: DICKSTEIN SHAPIRO MORIN & OSHINSKY LLP, 2101 L STREET NW, WASHINGTON, DC, 20037-1526, US

(21) Appl. No.: 10/292,476

(22) Filed: Nov. 13, 2002

Related U.S. Application Data
(60) Provisional application No. 60/331,209, filed on Nov. 13, 2001.

(51) Int. Cl. A61B 17/58

(52) U.S. Cl. 606/73; 606/80; 606/104

(57) ABSTRACT

A fixation device for securing soft tissue or suture to bone, such as in the case of rotator cuff repair and primary or secondary ACL tibial side fixation, is disclosed. The fixation device includes an implant screw and a washer that attaches to the head of the screw, yet allows the screw to be turned independently of the washer during insertion of the screw. The washer can be a cap-shaped washer, for covering and securing suture knot bundles around the implant screw, or a spiked cap, for engaging soft tissue. If necessary, the length of the implant screw may be cut to adjust its length.

This application claims the benefit of U.S. Provisional Application Serial No. 60/331,209, filed Nov. 13, 2001.

### FIELD OF THE INVENTION

The present invention relates to methods and apparatus for fixation of sutures and soft tissue to bone using an implant screw and washer assembly.

### BACKGROUND OF THE INVENTION

When soft tissue such as a ligament or a tendon becomes detached from a bone, surgery is usually required to reattach or reconstruct the tissue. In many cases, a tissue graft is attached to the bone to facilitate regrowth and permanent attachment. Various fixation devices, including sutures, screws, staples, wedges, and plugs have been used in the past to secure soft tissue to bone. In some prior fixation devices, a washer is used in conjunction with a bone screw whereby the washer provides additional means for securing soft tissue to the bone during and after insertion of the screw into the bone.

U.S. Pat. No. 5,718,706 discloses one example of a fixation screw/washer system. In this disclosed device, the screw rests on the outer surface of the washer and, thus the head of the screw and the washer must be countersunk below the surface level of the bone. Otherwise, the screw head would be proud to the bone after insertion, which could cause abrasion or irritation of the surrounding soft tissue. Also, if the screw and washer are used to secure suture attached to tissue, the suture is wrapped around the screw under the washer to thereby raise the washer and/or screw/washer assembly off the bone, which also leads to the problem of tissue abrasion.

Other examples of fixation screw and washer systems include U.S. Pat. Nos. 4,988,351 and 6,248,108, wherein the disclosed washers are formed with spikes to engage soft tissue to hold it to the bone. In each of these prior art systems, however, the washer loosely surrounds the shaft of the screw and readily shifts around both vertically and laterally, and also rotationally during insertion of the screw, unless the washer is integral with the screw. However, if the screw is integral with the washer, and the washer is designed to engage soft tissue, rotation of the screw is limited after engagement of the tissue.

### SUMMARY OF THE INVENTION

The method and fixation device of the present invention overcomes the deficiencies of the prior art noted above and provides an assembly for securing soft tissue or suture to bone. Examples of procedures in which the device and method of the present invention can be used include rotator cuff repair and primary or secondary ACL tibial side fixation.

The fixation device according to the present invention includes an implant screw and a washer that attaches to the head of the screw, and which allows the screw to be turned independently of the washer during insertion of the screw. The washer can be a cap-shaped washer for covering and securing suture knot bundles around the implant screw, or a spiked washer for engaging soft tissue. The screw can be provided in either a uni-cortical or bi-cortical design.

According to a first method of using the implant screw with the selected washer in accordance with the present invention, a uni-cortical implant screw is installed so that the distal end of the screw abuts the posterior cortex of a subject tibia and the shoulder of the screw is encased in the anterior cortex thereof. If necessary, the length of the screw may be cut to adjust its length.

A second method of using the implant screw and washer assembly of the present invention is similar to the first method, except that a bi-cortical screw is installed so that the distal end of the screw is inserted into the posterior cortex in addition to the proximal end of the screw being affixed in the anterior cortex of the bone.

Other features and advantages of the present invention will become apparent from the following description of the invention, which refers to the accompanying drawings.

### BRIEF DESCRIPTION OF THE DRAWINGS

FIG. 1 shows a perspective view of a first embodiment of an implant screw in accordance with the present invention.

FIG. 2 shows a proximal end view of the implant screw shown in FIG. 1.

FIG. 3 is a cross-sectional view of the implant screw of FIG. 2 taken along line 3-3′.

FIG. 3A is an enlarged view of the circled region of FIG. 3.

FIG. 4 shows an elevational view of a second embodiment of an implant screw in accordance with the present invention.

FIG. 5 shows a cross-sectional view of the implant screw of FIG. 4 taken along line 5-5′.

FIG. 5A is an enlarged view of the circled region A of FIG. 5 and illustrating a proximal view of the implant screw of FIG. 5.

FIG. 5B is an enlarged view of the circled region B of FIG. 5 and illustrating a distal view of the implant screw of FIG. 5.

FIG. 6 shows a proximal end view of the implant screw of FIG. 4.

FIG. 7 shows an elevational view of a third embodiment of an implant screw in accordance with the present invention.

FIG. 8 shows a cross-sectional view of the implant screw of FIG. 7 taken along line 8-8′.

FIG. 9 shows a proximal end view of the implant screw of FIG. 7.

FIG. 10 is a perspective view of a first embodiment of a washer in accordance with the present invention.

FIG. 11 shows a top view of the washer shown in FIG. 7.

FIG. 12 is a cross-sectional view of the washer of FIG. 8 taken along line 12-12′.

FIG. 13 shows a perspective view of a washer second embodiment in accordance with the present invention.

FIG. 14 shows a bottom elevational view of the washer shown in FIG. 13.

FIG. 15 is a cross-sectional view of the washer of FIG. 14 taken along line 15-15′.

FIG. 16 shows the implant screw of FIG. 1 in an assembled state with the washer shown in FIGS. 10-12.

FIG. 17 shows the implant screw of FIG. 1 in and assembled state with the washer shown in FIGS. 13-15.

FIG. 18 shows a ligament graft with an interference screw at an intermediary stage of insertion into a tibial and femoral tunnel, and which will undergo further fixation with an implant screw and washer in accordance with the present invention.

FIG. 19 shows the ligament graft of FIG. 18 further secured with an implant screw and washer in accordance with the present invention.

FIG. 20 illustrates a drill bit for preparing a bone hole in which a screw and washer in accordance with the present invention is to be installed.

FIG. 21 shows a driver used in conjunction with the present invention.

FIG. 22 shows a guide wire used in conjunction with the present invention.

FIG. 23 shows a bone tap used in conjunction with the present invention.

FIG. 23A is a detailed view of the distal end of the bone tap shown in FIG. 23.

FIG. 24 shows a countersink used in conjunction with the present invention.

FIG. 25 shows a top view of a screw cutting guide used in conjunction with the present invention.

FIG. 25A shows a side elevational view of the screw cutting guide of FIG. 25.

### DETAILED DESCRIPTION OF THE PREFERRED EMBODIMENTS

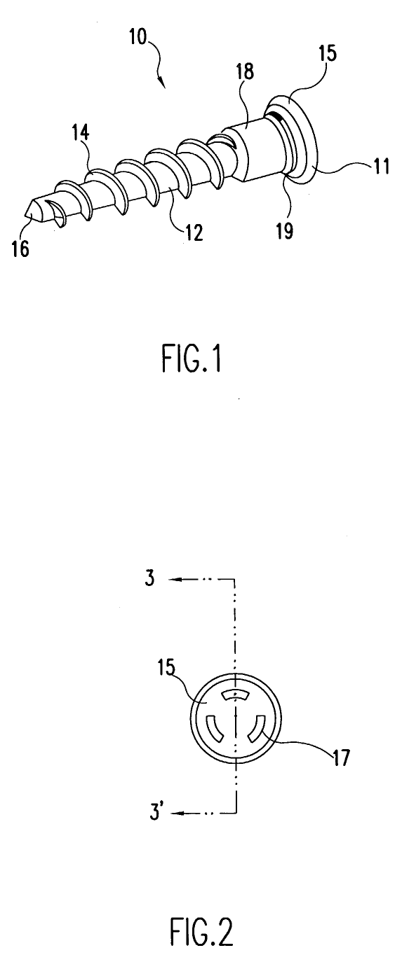

Referring now to the drawings, where like elements are illustrated by like reference numerals, FIG. 1 illustrates a first embodiment of implant screw 10 of the present invention, which includes a tapered body 12 having a continuous thread 14 and a sharpened point at its distal end 16. At proximal end 11, the implant screw 10 is provided with a shoulder section 18, a neck section 19 and a head 15, all illustrated in FIG. 1.

Preferably, the tapered body 12 has a length of about 35 mm (including the shoulder section and the proximal head 15), and tapers slightly, for example at a 4 degree slope, from a 6.35 mm proximal outer diameter (including the thread 14) toward the distal end 16 having the pointed tip. The proximal and distal surfaces of the thread 14 are angled equally from the body of the screw, and together form about a 60° angle. In the preferred embodiment, the pitch of the thread forms about six flights of thread along the tapered body 12.

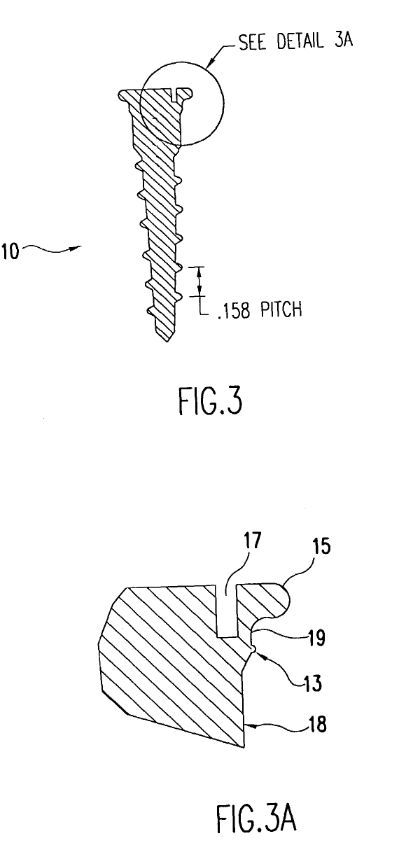

The shoulder section 18 is formed at the proximal end of the threaded section, and has no threads and a constant diameter approximately equal to the outer diameter of the proximal-most flight of thread on the tapered screw body 12. In the example illustrated in FIGS. 1 and 3, shoulder section 18 has a length of approximately 5 mm. The neck portion 19 is located at the proximal end of the shoulder portion 18 just distally of the head 15. The neck portion 19 has a diameter slightly wider than that of shoulder portion 18 and a height corresponding to a thickness of a washer to be attached to the screw, as will be described below. As shown in FIG. 3A, the neck portion 19 also has elongated bumps 13 formed on the radial surface of and along the distal-most end of neck portion 19, just proximally of the transition between the neck portion 19 and the diameter of shoulder portion 18. Preferably, the neck portion 19 comprises two elongated bumps spaced 180 degrees apart. The function of bumps 13 will be described in detail below.

Head 15 is approximately 10 mm in diameter and has a smooth, low thick profile, about 1.0 to 1.5 mm, to minimize trauma to surrounding tissue. The proximal face of the head 15 has at least one opening 17 (FIG. 2) for engaging a driver. Although many different drive coupling arrangements are possible, the preferred embodiment of the invention has three equally spaced openings or arcuate slots 17 formed in the disk-shaped head to engage the driver used for delivery and installation of the implant, as described below. Slots 17 are positioned in the central portion of head 15 which lies above neck portion 19 so that the depth of slots 17 can extend slightly below the depth of head 15 and into neck portion 19, as can be seen in FIGS. 3 and 3A. In this manner, slots 17 enable solid coupling between an engaged driver and the screw for driving the screw.

The implant screw 10 is preferably formed of a bioabsorbable, biocompatible material, such as Resomer L210 Poly (L-Lactide) acid (PLLA) or an equivalent material. In addition to being biocompatible and bioabsorbable, an implant screw formed of PLLA material provides the advantages of not being visible on radiographs and not interfering with MRI or CT scans. Also, the PLLA material allows the screw to be easily cut to a desired length with a rongeur, if needed, yet allowing the implant screw to be strong enough to provide a fixation strength of approximately 1000 Newtons and 90° shear force.

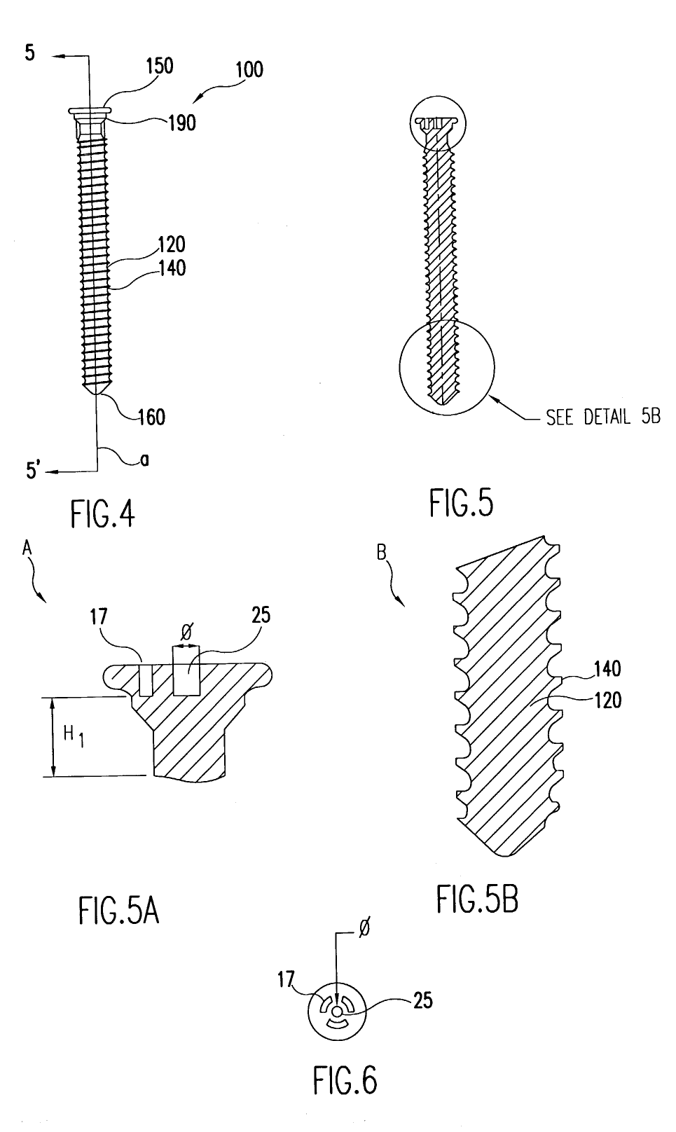

A second embodiment of the implant screw of the present invention, intended for bi-cortical fixation, rather than for uni-cortical fixation, is illustrated with reference to FIGS. 4-6. As shown in FIG. 4, implant screw 100 is similar to the implant screw 10 of the first embodiment, except that the screw body 120 is longer and is not tapered, and does not include a shoulder section. In this manner, threads 140 extend all the way proximally to neck portion 190.

The implant screw 100 is preferably provided in a 55 mm length, with about 30 flights of thread along body 120 and a height H₁ of unthreaded portion of the implant screw body 120 of about 10 to 15 millimeters. The implant screw 100 may be provided in a variety of sizes, including, but not limited to screws having a major diameter of approximately 2.7 mm, 3.5 mm, 4.5 mm, 4.0 mm and 6.5 mm. In these exemplary sizes of the implant screw 100, the preferred shaft lengths are respectively 24 mm, 40 mm, and 70 mm having cortical threads, and 50 mm and 110 mm having cancellous threads. Also, as illustrated in FIG. 5B, the angle between the distal surface of each thread flight and the proximal surface of the next thread flight in the distal direction is between approximately 30° to 40°. Of course, it should be understood that the implant screws of the types described in the present application may be manufactured or provided in additional or alternative sizes. Moreover, the implant screws exemplified herein may be provided having different measurements for any one or more of the dimensions mentioned above.

The tapering of the point at the distal tip 160 forms approximately a 45° with the vertical (longitudinal) axis “a” through the length of the implant screw. The thread 140 includes a gradual runout for approximately 2 to 3 flights at the proximal portion of the main body 120 just distally of neck portion 190.

As illustrated in FIGS. 5A and 6, in addition to slots 17, head 150 is provided with a central opening or bore 25 of about 1 to 2 mm deep, depending on the size of the implant screw. The central opening 25 is preferably circular, of diameter φ₁ (FIG. 5A) which is of about 3 to 5 millimeters for engaging a driver or a cutting guide, as described in more detail below. Optionally, in lieu of central opening 25 having a limited depth, the implant screw 100 may be cannulated throughout its entire length. Other features and characteristics of implant screw 100 are similar to the ones discussed above with respect to implant screw 10 shown in FIG. 1.

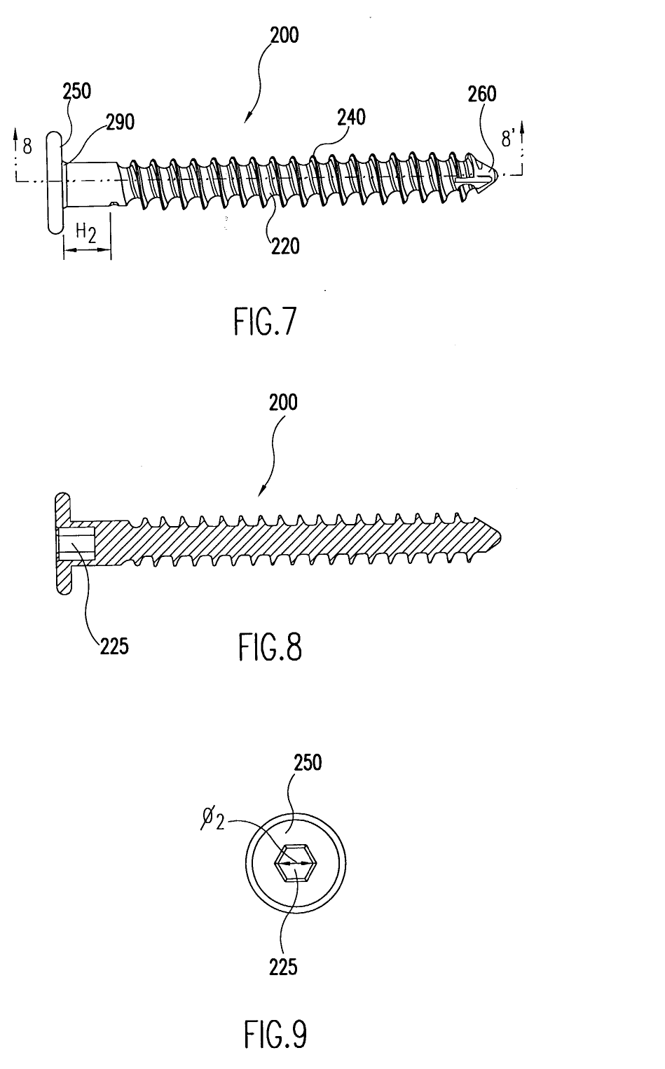

FIGS. 7-9 illustrate yet another embodiment of the present invention, according to which an implant screw 200 intended for bi-cortical fixation is formed of titanium or other compatible material, and not of bioabsorbable or biocompatible material. In addition, the implant screw 200 is provided with a central opening 225 (FIGS. 8 and 9) having a hexagonal configuration, and not a circular one as in the second embodiment described above with reference to FIGS. 4-6. The central opening 225 has a depth of about 2 to 8 millimeters and a diameter φ₂ (FIG. 9) of about 5 to 8 millimeters. The height H₂ (FIG. 7) of unthreaded portion of the screw body 220 is of about 10 to 15 millimeters.

As described below, implant screw 10, 100, 200 may be used in conjunction with a washer of the present invention, which can be fitted and retained around the neck portion 19, 190, 290 so that the washer is horizontally stable, yet can be rotated freely with respect to the implant screw 10, 100, 200. To achieve this capability, the diameter of neck portion 19, 190, 290 is sized to substantially correspond to the inner diameter of the washer so as to enable the washer to be rotated easily, without friction around the screw, while preventing the washer from tilting and shifting loosely around and along the shaft about when positioned around the neck portion 19, 190, 290. The diameter of neck portion 19, 190, 290 is also slightly smaller than the inner diameter of the washer to enable the washer to be rotated easily, without friction, around the screw.

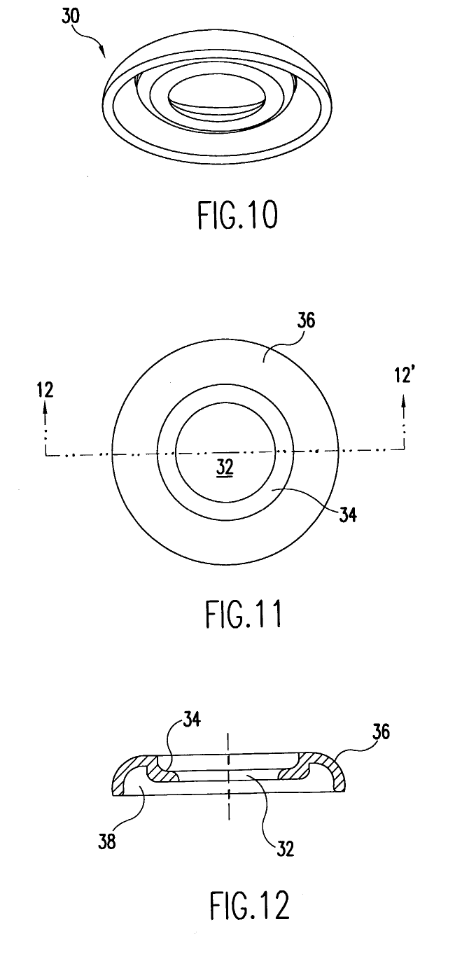

Two preferred embodiments of washers employed with a screw implant of the present invention, for example with the implant screw 10 of FIG. 1, are described below with reference to FIGS. 10-15.

A first preferred embodiment of a washer of the present invention which can be fitted onto the implant screw 10 is illustrated in FIGS. 10-12. Cap-shaped washer 30 has an outer diameter of about 10 to 16 mm and a central opening 32 slightly larger in diameter than the diameter of the neck portion 19 of the corresponding sized screw onto which the washer may be fitted.

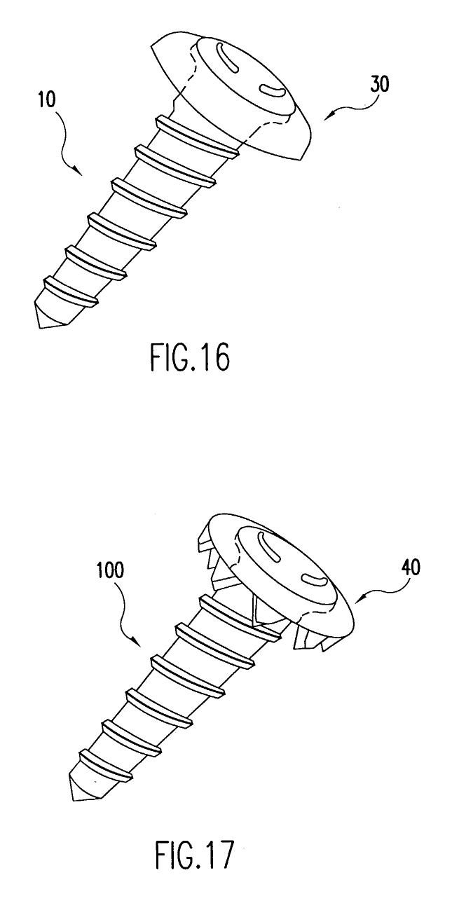

The cap-shaped washer 30 is also provided with a ledge 34 formed around the inner peripheral surface of washer 30 for seating the screw head 15 thereon, and a cap-shaped outer portion 36 rising above the level of and surrounding the seating ledge 34. Cap-shaped outer portion 36 is shaped so that, when washer 30 is positioned around the neck portion 19, the proximal surface of screw head 15 is substantially flush with the curved proximal surface of the cupped outer ring 36, as illustrated in FIG. 16. The height of cap-shaped outer portion 36 is approximately 3 mm to permit the formation of a space 38 (FIG. 12) under the curved surface of the cap-shaped portion 36 for covering suture knots tied around the periphery of the implant screw 10. Cap-shaped washer 30 thus provides the advantage of eliminating potential irritation to soft tissue incurred by the suture knots.

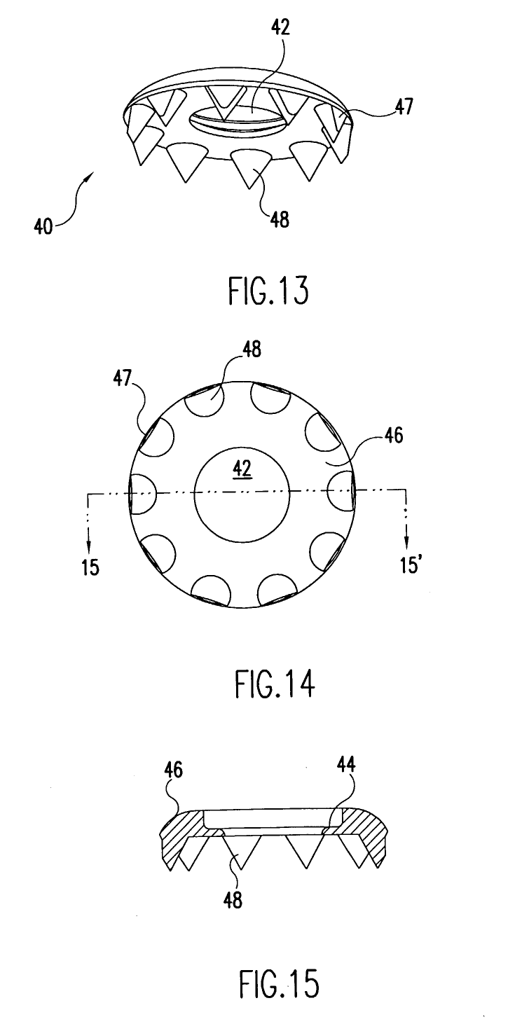

FIGS. 13-15 illustrate a second preferred embodiment of a washer of the present invention employed in conjunction with the implant screws described above. Spiked washer 40 is provided with a central opening 42 for receiving the implant screw 10 therethrough, a ledge 44 (FIG. 15) formed around the inner peripheral surface of washer 40, and an outer ring portion 46 encircling ledge 44. The inner and outer diameters of washer 40 are the same as those of washer 30 shown in FIGS. 10-12. Outer ring portion 46 has a rounded upper surface and a height greater than that of ledge 44. A plurality of protuberances or teeth 48 project downwardly from, and are arranged around, the periphery of the bottom surface of outer ring portion 46. As illustrated in FIG. 17, when the washer 40 is positioned around the neck portion 19, head 15 of the implant screw 10 is seated on the ledge 44 so that the proximal surface of the screw head 15 is substantially flush with the rounded proximal surface of table 46.

In the preferred embodiment, washer 40 has 10 teeth equally spaced around the perimeter of the bottom surface of the washer (FIG. 14), and each tooth 48 forms about a 60° conical point. While not critical to the invention, it is also preferred that the protuberances or teeth are round at their bases except for the radially-outward facing surfaces 47 of the same, which are provided with a sheared-off appearance to be substantially even with the outer circumferential edge of outer ring portion 46. Using this embodiment of washer 40, protuberances or teeth 48 engage the soft tissue to be secured in a stationary manner while the implant screw 10 is being installed into the bone.

Referring now to FIGS. 16-17, the selected washer is fitted onto the neck portion 19, 190, 290 of implant screw 10, 100, 200 by inserting the distal end 16, 160, 260 of screw 10, 100, 200 through the central opening of the washer from the top side thereof, and raising the washer relative to the screw. With reference to the implant screw 10, for example, the diameter of neck portion 19 is augmented slightly by the presence of elongated bumps 13 to thereby correspond with the inner diameter of the washer, so that friction is encountered when the washer is raised on the screw to this point. By pressing the two pieces together with additional pressure, the washer can be forced over the bumps and snapped into place around neck portion 19 above the elongated bumps 13 and below the head 15 of the implant screw 10. The washer is thus retained in position surrounding head 15 by the elongated bumps 13 and prevented from slipping off of neck portion 13. Between elongated bumps 13 and the bottom surface of head 15, neck portion 19 thus has a height which is at least equal to the thickness of the washer at the inner diameter thereof.

The implant screw and washer assembly of the present invention can be used to secure soft tissue in procedures such as ACL or PCL reconstruction, medial or lateral collateral ligament repair, patellar tendon repair, posterior oblique ligament repair and iliotibial band tenodesis procedures. An example of using the implant screw and washer assembly in accordance with the embodiments of the present invention to affix soft tissue, such as an anterior cruciate ligament (ACL), for example, to a tibia tunnel, will be discussed below.

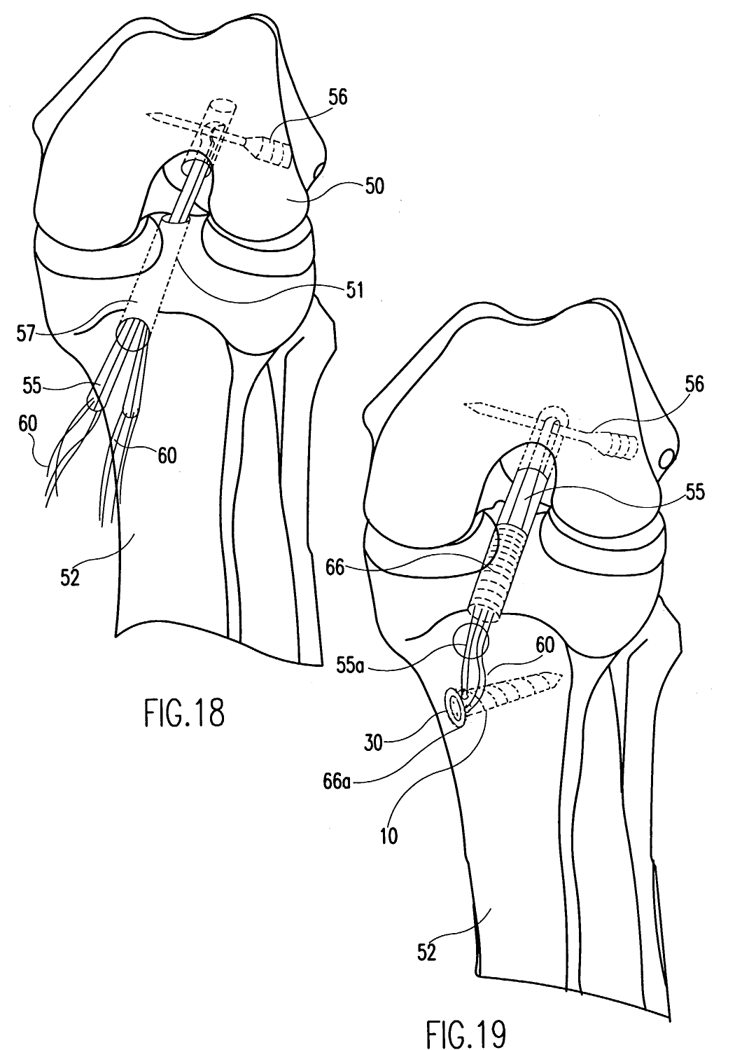

Reference in now made to FIGS. 18 and 19, which illustrate a ligament graft fixated with an interference screw in anterior cruciate ligament (ACL) reconstruction at intermediary (FIG. 18) and complete (FIG. 19) stages of insertion into a tibial and femoral tunnel, which will undergo further fixation with an implant screw and washer assembly of the present invention. As shown in FIG. 18, a tendon or ligament graft 55 having at least one attached strand of suture 60 at its distal end is partially inserted into longitudinal tibial tunnel 57 of tibia 52. FIG. 19 illustrates the tendon 55 of FIG. 18 loaded through the longitudinal tibial tunnel 57 and into a femoral socket of femur 50, and fixated with a transverse implant 56 and an interference screw 66.

In preparing to use an implant screw of the present invention, for example the implant screws 10, 100, 200 described in detail above, with a selected washer of the present invention, for example the washers 30, 40 also described above, a hole is drilled on the anterior tibia 52 and inferior to the tibial tunnel exit 55a where the implant screw of the present invention is to be installed.

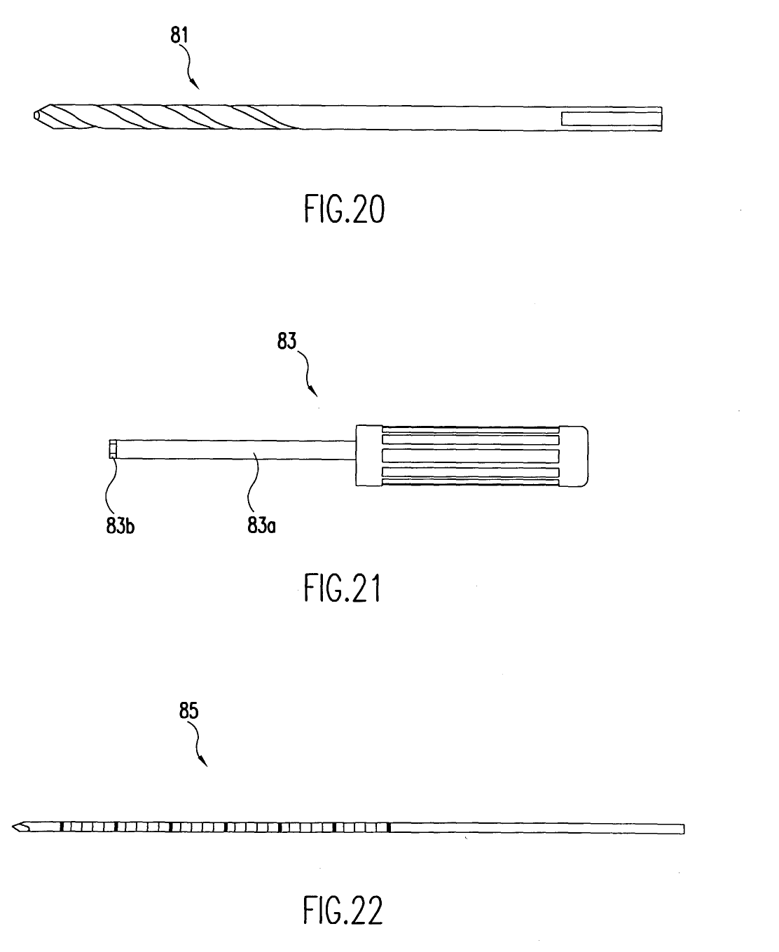

The tibia hole is formed by using a drill pin 81 illustrated in FIG. 20 which has a major diameter (the outer diameter across the threads) corresponding to the minor diameter of the screw to be inserted. Thus, for the uni-cortical implant screw 10 exemplified above with reference to FIG. 1, a 2.4 mm drill pin, corresponding to the minor diameter of the screw at the distal end of the tapered body 12, is used to install the uni-cortical implant screw 10. The drill pin 81 is advanced until the distal tip of the drill pin contacts the posterior tibial cortex. During the drilling of the hole, it is important to ensure that the drill pin 81 is perpendicular to the tibia 52.

Subsequent to drilling, the anterior cortex surrounding the hole must be decorticated using a counterbore drill tip 89, such as that shown in FIG. 24. Failure to decorticate the anterior tibial cortex may result in the structural failure of the implant fixation device due to the excess torque which must be applied thereto during insertion.

The counterbore drill tip 89 should be advanced one to two revolutions or until an adequate recess have been made for the head 15 of the implant screw 10. Similarly to the drill bit 81, counterbore drill tip 89 has a proximal end 89a constructed to be releasably engaged with a chuck in a quick-connect handle. Preferably, counterbore drill tip 89 also has a central cannula extending through the length of the drill tip from an opening at the proximal end to an opening at the distal end thereof between cutter edges 89b to thereby receive insertion of the drill pin to facilitate proper alignment during decortication.

A depth guage 85 such as the one illustrated in FIG. 22 is then inserted into the drilled hole to determine the length of the implant screw needed. The tip of the depth gauge should engage the bottom of the drilled hole. The depth of the hole is read to the top of the countersunk hole, or even with the bone surface.

Using the screw cutting guide 91 (FIGS. 25 and 25A), the selected implant screw, if bioabsorbable, is cut to a length corresponding to the depth measurement obtained for the drilled hole in accordance with the procedures described above. Cutting of the implant screws of the present invention to obtain cut-to-length implant screws may be conducted by employing a method and corresponding tool instruments such as the ones described in U.S. patent application Ser. No. 10/163,303 filed Jun. 7, 2002, the disclosure of which is incorporated by reference herein. As detailed in U.S. patent application Ser. No. 10/163,303, the length of screws may be adjusted during a fixation operation by employing a cutting guide or jig having a measuring block which is slidable along a rail adjacent a measuring scale and a holding block, to facilitate cutting the distal end of the screw once the desired length has been determined. A tip sharpener may be also incorporated into the cutting guide or may be provided separately, for reforming a point at the distal tip of the just-cut screw.

According to U.S. patent application Ser. No. 10/163,303, a preferred method for providing a cut-to-length screw includes selecting an appropriately sized fixation screw to be installed; drilling a hole across the fracture site using an appropriately sized drill bit; forming a countersunk bore across the drilled hole; inserting a measuring tool into the drilled hole to determine the length thereof; tapping either the entire length of the drilled hole or only the distal fragment thereof when the lag technique is to be performed; placing the selected fixation screw into a cutting guide; setting the cutting guide to a measurement corresponding to the measured length of the drilled hole; if the selected fixation screw is longer than the measurement set in the cutting guide; cutting off the excess length from the distal end of the screw; inserting the distal end of the cut fixation screw into the sharpener; and turning the screw until a pointed tip has been reformed at the distal end of the screw.

Subsequent to the cutting-to-length operation, the revised implant screw is then placed onto the end of an appropriately sized driver 83 (FIG. 21) and aligned with the drilled hole at the implant site. The driver is rotated to advance the screw into the hole until it is flush with or countersunk with the surface of the tibia 52.

Driver 83 includes a drive shaft 83a which may or may not be cannulated, depending on need for the procedure or if the fixation screw is also cannulated. In one embodiment, the distal end of drive shaft 83a includes three arcuate projections 83b for engaging the arcuate slots 17 formed in the head 15 of the implant screw 10, 100, as shown in FIG. 21. If the drive shaft is not cannulated, the distal end of the shaft preferably also includes a protruding nub for engaging the central bore 25 at the proximal face of the head of the implant screw. This design provides maximum insertion torque while alleviating the potential for stripping of the driver coupling structure in the screw. Of course, if the screw head of the implant screw is formed with an alternative drive coupling arrangement other than the three arcuate slots 17, for example, with a central opening with a geometry other than circular such as the hexagonal opening 225 of the implant screw 200 of FIGS. 7-9, the driver 83 is configured to have a matingly shaped drive head. Drive shaft 83a may include a quick-connect fitting at its proximal end for being fitted into a ratcheting handle, or may be attached to a non-ratcheting handle.

With the drill pin still in the fully inserted position in the just-drilled hole, the depth of the cancellous bone is measured by marking the drill pin at the intersection of the anterior tibial surface with the drill pin, such as by securely fasting a clamp or similar device to the drill pin at the intersection point. The drill pin is then removed from the hole with the clamp attached, and the depth of the hole via the marked drill pin is compared to the length of the implant. Since the threads of the screw will not properly grasp the cancellous bone if the distal tip of the screw contacts the posterior cortex, up to 10 mm may be cut from the distal end of the implant, if necessary, to enable full insertion of the implant.

Either the spiked washer or the cap-shaped washer is selected for assembly with the implant screw. The selected washer is snapped into place under the screw head 15, and the implant is advanced into the hole using the appropriate driver. If desired, however, the fixation screw can be used without any washer.

If the cap-shaped washer is used with the implant screw, about 2 to 3 mm of the shaft is left exposed above the tibial surface. At this time, graft traction sutures 60 (FIG. 19) are tied and/or knotted around the exposed implant shaft to form knots and/or knot bundles 66a (FIG. 19). If multiple knots are created, the different knot bundles are distributed around the shaft of the implant.

If the spiked washer is used, the screw may be inserted either through or adjacent to the soft tissue which is then wrapped under the washer. It should be noted that the thickness of the graft will impact the purchase of the screw into the bone and the security with which the graft is held against the bone by the spikes on the washer.

The implant is driven further into the hole until the washer and screw head are fully installed without overtightening. When properly and fully installed, the distal end of the screw abuts the posterior cortex of a subject tibia and the shoulder of the screw is encased in the anterior cortex thereof. If the cap-shaped washer is used, knot bundles 66a (FIG. 19) are captured under the cap-shaped outer ring portion of the washer 30. If the spiked washer is used, the screw is inserted through or adjacent to the soft tissue and the screw is advanced until the spikes engages the ligament and securely affixes it to the bone. Since the implant is constructed to rotate independently of the attached washer, the soft tissue can be securely engaged by the spiked washer without tearing.

As mentioned above, the surgeon should take care not to overtighten the implant in the hole. Also, it should be noted that the implant and/or the driver may be damaged if the driver is not fully seated in the drive slots formed in the implant head, or if the driver is misaligned in the implant head.

In the procedure for installing a bi-cortical screw such as that described above with reference to bi-cortical implant screw 100 of the second embodiment of the invention, a 4.2 mm drill pin is used to drill through the anterior cortex, the cancellous bone, and just through the posterior cortex. As with the installation procedure for the uni-cortical screw, the anterior cortex surrounding the drilled hole is decorticated using the counterbore drill tip, and the drill pin is marked once the distal end thereof penetrates through the posterior cortex, so that the correct length of the bicortical screw can be determined. Ideally, the length of the screw should be such that the end of the screw extends just through the posterior cortex. The screw should not extend too far out of the cortex otherwise damage to other tissue may occur. Again, the screw can be cut to the desired length, if necessary.

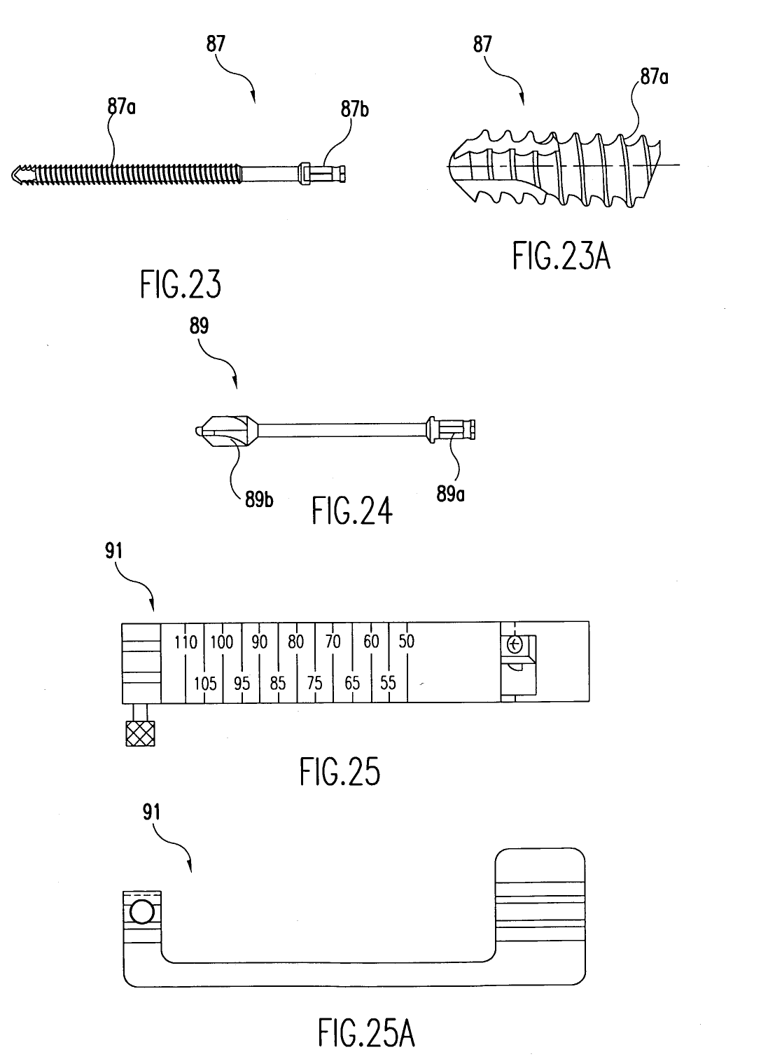

When a bi-cortical screw such as the implant screw 100 described above with reference to FIG. 4 is to be inserted, a tap must be used to enable penetration of the resorbable screw into the hard material of the posterior cortex. Tap 87 is illustrated in FIGS. 23 and 23A as having a threaded body 87a which is at least as long as the length of the implant screw 100 to be inserted. The major and minor diameters of threaded body 87a are equal to or slightly smaller than the major and minor diameters of the screw 100. In the example shown in FIGS. 23 and 23A, the major diameter is 6.3 mm and the minor diameter is 4.2 mm. The proximal end of tap 87 may include a quick-connect fitting 87b (FIG. 23) for being fitted into a ratcheting handle, or tap 87 may be simply attached to a non-ratcheting handle as conventionally known in the art.

Next, the tap 87 is used to further define the hole through the posterior cortex prior to insertion of the bicortical screw. The steps for assembling a selected washer with the screw and for securing sutures, suture knots, and soft tissue with the screw and washer combination are the same as with the procedure using the uni-cortical screw. With screw threads in the posterior cortex, however, the bi-cortical screw provides a more secure fixation of the soft tissue than that provided by the uni-cortical screw.

Although the embodiments of the present invention have been described above with reference to an implant screw having a circular or hexagonal central opening formed within the head of the screw, such as central openings 25 and 225, the invention is not limited to these embodiments. Accordingly, the invention contemplates implant screws having central openings of various geometrical shapes and configurations, depending on the drive coupling arrangements, and as desired. In addition, although the present invention has been described above with reference to only one implant screw with one washer attached as part of a side tibial fixation operation, the invention also contemplates the use of a plurality of such implant screws with or without corresponding washers attached.

Although the present invention has been described in relation to particular embodiments thereof, many other variations and modifications and other uses will become apparent to those skilled in the art. It is preferred, therefore, that the present invention be limited not by the specific disclosure herein, but only by the appended claims.

What is claimed is:

1. A fixation device comprising:
a bioabsorbable screw comprising a distal end, a body portion of a first diameter and a proximal end, wherein at least a portion of said body portion is threaded, and wherein said proximal end is provided with a head of a second diameter, said proximal end being integrally attached to said head; and
a bioabsorbable washer removably connected to said bioabsorbable screw and having an opening of a third diameter, said third diameter being greater than said first diameter but smaller than said second diameter.

2. The fixation device of claim 1, wherein said bioabsorbable screw is formed of PLLA material.

3. The fixation device of claim 1, wherein said bioabsorbable washer is formed of PLLA material.

4. The fixation device of claim 1, wherein a proximal region of said body portion having a predetermined height is not threaded.

5. The fixation device of claim 1, wherein said washer has a proximal face and a distal face.

6. The fixation device of claim 5, wherein said distal face is provided with a plurality of protuberances.

7. The fixation device of claim 6, wherein said protuberances have a triangular cross-section.

8. The fixation device of claim 1, wherein said washer has a circular configuration.

9. The fixation device of claim 8, wherein said washer is provided with a ledge for supporting said head of said screw.

10. The fixation device of claim 9, wherein said washer is further provided with an outer cap-shaped portion.

11. The fixation device of claim 10, wherein said outer cap-shaped portion of said washer forms a space covering suture knots.

12. The fixation device of claim 10, wherein a proximal surface of said head is flush with a proximal surface of said washer.

13. The fixation device of claim 1, wherein said bioabsorbable screw is a uni-cortical screw.

14. The fixation device of claim 1, wherein said bioabsorbable screw is a bi-cortical screw.

15. A bioabsorbable surgical implant for supporting tissue in a predetermined position in a body, comprising:
a bioabsorbable screw comprising a distal end, a shaft of a first diameter, and a proximal end, wherein at least a portion of said shaft is threaded, and wherein said proximal end is provided with a head of a second diameter, said proximal end being integrally attached to said head; and
a bioabsorbable washer having a central opening dimensioned to fit said shaft, said central opening having a third diameter which is greater than said first diameter but smaller than said second diameter.

16. A surgical implant for supporting tissue in a predetermined position in a body, comprising:
a titanium screw comprising a distal end, a shaft of a first diameter and a proximal end, wherein at least a portion of said shaft is threaded, and wherein said proximal end is provided with a head of a second diameter, said proximal end being integrally attached to said head, said head being provided with a first central opening of hexagonal configuration; and
a washer having a second central opening dimensioned to fit said shaft, said second central opening having a third diameter which is greater than said first diameter but smaller than said second diameter.

17. A method for performing a surgical bone fixation procedure, said method comprising the acts of:
drilling a hole across a bone site;
selecting a bioabsorbable implant screw having a predefined diameter;
measuring said drilled hole to determine a necessary length for said implant screw;
tapping said drilled hole;
cutting said implant screw to said necessary length to form a cut-to-length implant screw;
sharpening a distal end of said cut-to-length implant screw to form a pointed tip;
attaching a washer around said head of said cut-to-length implant screw prior to driving said cut-to-length implant screw into said drilled hole; and
driving said cut-to-length implant screw into said drilled hole.

18. The method of claim 17, wherein said washer is a bioabsorbable washer.

19. The method of claim 17, wherein said implant screw is a bicortical screw.

20. The method of claim 17, wherein said act of cutting said implant screw comprises providing a cutting guide which is appropriately sized for receiving said implant screw.
